(19) United States
(12) Patent Application Publication
(10) Pub. No.: US 2006/0111705 A1
(43) Pub. Date: May 25, 2006

(54) Device for destruction of tumor tissue

(75) Inventors: Gerhard Janzen, Wohnsitz (DE); Buu-Son Trinh, Wohnsitz (DE); Holger Soring, Wohnsitz (DE); Jorg Soring, Wohnsitz (DE)

Correspondence Address: HORST M. KASPER, 13 FOREST DRIVE, WARREN, NJ, 07059, US

(73) Assignee: SORING GMBH

(21) Appl. No.: 11/285,547

(22) Filed: Nov. 21, 2005

(30) Foreign Application Priority Data
Nov. 19, 2004 (DE) 10 2004 055 866.3

(51) Int. Cl. A61B 18/14

(52) U.S. Cl. 606/41

(57) ABSTRACT

An instrument for electrochemical treatment of the human body or of an animal body is disclosed. The instrument is useful for the destruction of tumor tissues. One or several electrodes are furnished for insertion into the body to be treated. A direct current generator (1) generates a DC voltage. Connection lines (2,3,3′) for the generated direct current power are connectable to the generator. A switching box (9) inserted into the connection lines for a selection of electrode polarity. At least one trocar sleeve (7,7′) is connected with a first end to the connection lines., At least one trocar thorn (12) is insertable into the trocar sleeve. At least one electrode (5,5′) is insertable into the trocar sleeve such that an end of the electrode (5,5′) protrudes from a second end of the trocar sleeve. Tumors having sizes up to about 50 centimeters diameter can be treated with the instrument.

### BACKGROUND OF THE INVENTION

1. Field of the Invention

The present Invention concerns an apparatus for the electrochemical treatment of a human body or of an animal body, in particular for the destruction of tumor tissues with one or several electrodes for introduction into the body to be treated, wherein the electrodes are disposed at a distance relative to each other.

2. Brief Description of the Background of the Invention Including Prior Art

Various methods and apparatuses for the destruction of tumor tissues have become known. For example according to U.S. Pat. No. 4,565,200 high frequency technologies are employed in order to coagulate tissues and/or to separate tissues. Preferably a frequency region from 300 kHz to 2 MHz is employed here. This pre-known bipolar electrode arrangement comprises two parallel disposed needle electrodes, wherein the needle electrodes are being stung into the tissue, and whereby the tissue disposed between the electrodes is heated based on the current flow and therewith coagulated.

The German printed Gebrauchsmuster DE 20003952 U1 describes a sonde, which is connected with a connector to a generator by way of a plug connection. The sonde comprises a flexible part and stiff parts successively following to the flexible part, wherein the electrodes are disposed at the end of the stiff parts. The overall construction provides only limited conveniences to the practitioner physician.

The European patent document EP 0871405 B1 teaches a bipolar high frequency surgical instrument. An electrically not conducting spacer or holder is provided, wherein the distance holder is brought in between the electrodes, that is between the claws of the tongue during a cutting of tissue. At least one channel is furnished, which serves as a flushing and/or suction channel.

Werner Seifermann in the journal Onkologie, issue 9, year 2004, p. 33 describes bio Electro tumor therapy. A self focusing current is driven through the tumor by way of an exact electrode arrangement. The article alleges that the electrical resistance of the tumor is 10 times lower as compared to the healthy tissue and consequently the current focuses in the tumor and polarizes the tumor. HCl is generated by this electrolysis and damages the cell membranes of the tumor tissue.

A high frequency therapy device is also described in the European patent 0714635 B1, wherein frequencies from 300 kHz to 1 MHz are employed, wherein temperatures in the region of from 60 to 100 degrees centigrade are generated in the region of the electrode tip for coagulation or, respectively, necrotization of the tissues.

These so-called thermal treatment methods are associated with the disadvantage that the complete body of the patient is subjected to this alternating voltage.

### SUMMARY OF THE INVENTION

1. Purposes of the Invention

Therefore, it is an object of the present Invention to create an instrument and a method for the destruction of tumor tissues, wherein the electrical load of the patient is small and wherein no thermal load occurs.

It is another object of the invention to provide a reliable instrument for necrotization of tumor tissue based on electrolyzing.

These and other objects and advantages of the present invention will become evident from the description which follows.

2. Brief Description of the Invention

The present invention provides

This object is achieved starting from an arrangement for electrochemical treatment of the human body or of an animal body, in particular for the destruction of tumor tissues with one or several electrodes for insertion into the body to be treated, and with the instrument of the present invention comprising a generator (1) for generating a DC voltage, connection lines (2,3,3′) connected to the generator (1), at least one trocar sleeve′ (7,7) attached to one of the connection lines, at least one trocar thorn (12) to be inserted into the trocar sleeve, and at least one electrode (5,5′) to be connected to one of the connection lines and to be inserted into the trocar sleeve.

It is possible to destroy liver tumors electrochemically with the aid of the Invention instrument and the therewith applicable method while employing the today wide spread minimally invasive operation method. A DC current generated by a generator is led through special platinum electrodes into the tumor of the respective organ according to this method. A precise placement of these electrodes can be controlled by way of x-ray measurements or by ultrasound measurements.

The DC current, which flows between two or several platinum electrodes, leads to a destruction of tissue by way of electrolysis. A substantial pH value shifting occurs in the issue based on the ionic migration. A dissociation occurs. An acid pH value (acidosis) and chlorine gas is formed at the anode. An alkaline pH value (alkalosis) is generated at the cathode and hydrogen gas is formed. The pH values are disposed outside of the physiological region and are damaging to tissue. The DC current also leads to a change of the membrane potentials by changing the electrolyte environment around the cell and in the cell. Important physiological functions as for example the potassium-sodium-pump are disturbed by the change of the membrane potentials, that is the change of a basic environment to an acid environment effects the formation of necrosis. Various salts disassociate into cations and anions in an electrical field such that the homoeostasis and biological equilibrium of the cell is lifted. The tumor tissue is devitalized.

The electrical devitalization is no usual electrical injury. An expulsion of the electricly induced necrosis takes place only after some time. The losses of substance correspond to the original spread of the tumor tissue by size, that is depending on the size of the tumor different multitudes of electrodes are required.

This non-thermal method improves the compatibility of the treatment generally based on the lack of temperature variations and permits the performance of the treatment under local anaesthesis. A further advantage is furnished in that the electrical current does not flow through the body of the patient. The operation and action of the DC current takes place only between the electrodes.

It is a further advantage of this method that the transcutaneous minimally invasive treatment of non-resectable tumor is possible with the cyto reduction.

An application of the electrodes occurs mostly percutaneously under local anaesthesis. A fitting trocar is led up to the tumor for this purpose. The electrode is then positioned in the trocar. The positioning of the electrodes should be continuously monitored through for example ultrasound instruments.

Bipolar electrodes are employed at sizes of tumors from 10 to 20 mm diameter. The bipolar electrode is led into the middle of the tumor with the aid of a trocar, such that the active electrode part is disposed in the middle third of the metastases.

Mono polar electrodes are employed with the tumor sizes from 10 to 50 mm. Mono polar electrodes are inserted into the tumor with the aid of a trocar at the boundary of the medium third of the tumor. The active part is adjusted (one-third of the diameter of the tumor). The distance between the electrodes should amount to at least 15 mm. The electrodes should always be inserted into the tumor with the aid of picture furnishing ultrasound methods (for example C-arc). Since the tumor tissue exhibits a much lesser electrical internal resistance as compared to healthy tissue, healthy tissue is not damage upon careful adjustment of the individual values. Healthy tissue exhibits and about 10 times higher electrical resistance as compared to tumor tissue.

The novel features which are considered as characteristic for the invention are set forth in the appended claims. The invention itself, however, both as to its construction and its method of operation, together with additional objects and advantages thereof, will be best understood from the following description of specific embodiments when read in connection with the accompanying drawing.

### BRIEF DESCRIPTION OF THE DRAWING

The Invention is in the following by way of example and by way of drawings described; there is shown:

FIG. 1: an illustration of hydrolysis taking place in a rock salt solution,

FIG. 2: an apparatus arrangement without switching box inclusive trocar sleeve, adjustment element and electrode;

FIG. 3: an instrument arrangement with switching box, inclusive trocar sleeve, adjustment element, table and electrode;

FIG. 4a: the arrangement trocar sleeve, adjustment element, and trocar thorn;

FIG. 4b: the arrangement trocar sleeve, adjustment element, and electrode;

FIG. 5: a liver with tumor and bipolar electrode;

FIG. 6: a liver with tumor and mono polar electrodes;

FIG. 7 a liver with a tumor and a polar electrode positioned in the healthy tissue and a second polar electrode positioned in the tumor;

FIG. 8 a liver with the tumor and two mono polar electrodes positioned outside of the tumor near two oppositely disposed peripheries of the tumor;

FIG. 9 is a schematic diagram showing the construction of the trocar;

FIG. 10 is a front elevational view of the generator apparatus;

FIG. 11 is a rear elevational view of the generator apparatus;

FIG. 12 is a schematic block circuit diagram of the current generator and the voltage automatic control;

FIG. 13 is a schematic block circuit diagram of the voltage automatic control;

FIG. 14 is a schematic block circuit diagram of the current generator.

### DESCRIPTION OF INVENTION AND PREFERRED EMBODIMENT

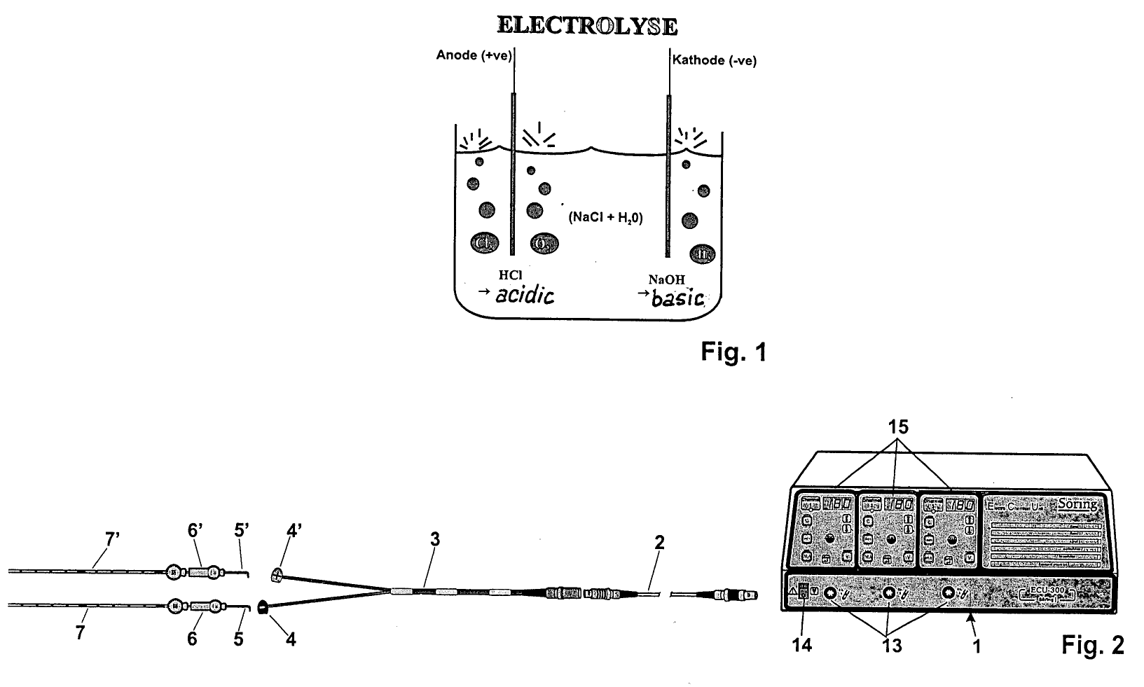

The perspective view shown in FIG. 1 clearly demonstrates that after application of a DC voltage to a rock salt solution, this rock salt solution is decomposed into HCl and sodium hydroxide by the electrolysis method. An acid pH value is generated at the anode and chlorine gas is formed. An alkaline pH value is generated at the cathode and hydrogen gas is formed. The present Invention is built on this basic knowledge.

A complete instrument is illustrated in FIG. 2. The generator (1) is furnished with three functional units (15) and the associated connection bushings (13). In addition, a power switch (14) is furnished and the instrument is connected to the electrical grid by the power switch (14).

The parameters required for the treatment can be separately adjusted for the individual connection bushings. Depending on the case of application—as can be recognized from FIG. 2—two electrodes (5,5′) can be connected to the connection bushings of the generator (1) by way of the connection cable (2) and the plug attached thereto. The instrument cable (3) is connected to this connection cable (2), wherein the plugs (4,4′) are attached at the other end of the instrument cable (3). These plugs (4,4′) are electricly conducting connected to the electrodes after the insertion and the precise fixation of the trocar sleeves (7,7′) and after the insertion of the electrodes (5,5′) into the respective trocar sleeve (7,7′). Depending on which electrode length is available, an adjustment element (6,6′)′ can be disposed between the trocar sleeve (7,7′) and the plug (4,4′).

The length of the electrodes depends on the field of application. Depending on the location of the tumor in the body, the corresponding trocar sleeve (7) and coordinated trocar thorn (12) and needle electrode (5) are selected of an appropriate length. The length of the free electrode part depends on the size or, respectively, the thickness of the tumor.

The hook shaped angle of the needle electrode serves as a stop and for a safe contacting with the plug element. The needle electrode is inserted into the trocar sleeve through the adjustment element (6) and the handle part (17).

One trocar thorn (12) is inserted in a trocar sleeve (7) for safe positioning of the electrodes (5,5′) in the tumor and the trocar thorn (12) is screwed onto the thread (18) of the handle part (17) by way of a thread formed into the handle. The tip of the trocar thorn (12) is ground very sharp such that the units trocar sleeve/trocar thorn can be shifted relatively easy through the skin. The surgeon can place the tip of the trocar thorn very precisely in the tumor under control by ultrasound or x-rays. If the predetermined location has been reached then the surgeon pulls the trocar thorn (12) out of the trocar sleeve (7) and then shifts the electrode (5) into the trocar sleeve (7). Since the electrode (5) and the trocar thorn (12) exhibit the same length, the electrical connection to the generator (1) can be produced after the fixation of the electrode. It is sufficient to employ two electrodes in case of smaller tumors. If however a larger tumor is concerned, then four or more electrodes have to be placed in the tumor. The instrument cable (3,3′) is furnished with several plugs (4,4′) corresponding to the number of required electrodes. If the polarity has been fixed for the plugs (4,4′), then the plugs (4,4′) are designated respectively with “+” and “−”. Of course, also a color designation or marking with numbers and/or letters is possible.

Preferably the cables are braided copper cables. The connections provided are plug connections. It is not important at which end the plugs and the sockets are disposed. Preferably sockets are provided on the generator side and plugs are provided at the side of the electrodes.

The trocar sleeve (7,7′) is also shown in FIG. 2 but may be more difficult to recognize. The construction of the whole press the can be recognized more clearly from FIGS. 4a and 4b. The figures show that the trocar sleeve and the adjustment element are connected initially to each other. Then following the trocar thorn (12) is the slipped through the trocar sleeve (7) in order to stitch an opening into the tissue. Thereafter the trocar thorn (12) is pulled out and the needle electrode (5) is inserted.

A special securing of the trocar sleeve (5) is not required. The physician observes the markings at the trocar sleeve (7) and holds the trocar sleeve (7) with one hand by using the other hand for changing the electrode (5) or the trocar thorn (12).

The alignment of the electrodes by the physician is performed under ultrasonic control or under MRT-or CT-monitoring. If desired the physician can pre-drill a hole with the biopsy set and then can employ the trocar sleeve or isolating sleeve.

The trocar sleeve (7) is preferably a straight sleeve. The trocar sleeve can also be bent. Advancing a trocar sleeve (7) would require that the trocar thorn (12) and also the needle electrode (5) are formed from a flexible material. Wire platinum is a relatively soft metal, nevertheless a straight electrode can be handled easier as compared to a bent electrode.

The electrode (5) is made out of platinum or at least in the region, which comes into contact with the tumor tissue, is made of platinum. The trocar thorn (12) comprises stainless-steel or a titanium alloy or titanium and the trocar sleeve (7) comprises a plastic which can be cleaned in an autoclave such as for example poly tetra fluoro ethylene or PEEK MT. The trocar sleeve 7 is preferably made out of an electricly nonconducting material such as for example poly tetra fluoro ethylene or PEEK MT. Liquids can be fed into the tumor through the trocar sleeve (7).

The adjustment element serves for adjusting the part of the needle electrode protruding from the trocar sleeve. The adjustment element can also be designated as a spacer piece. If for example the needle electrode is to protrude by 10 mm from the front and of the trocar sleeve, then the 10 mm adjustment element is inserted; if the electrode is to protrude by 30 mm, then the 30 mm element is employed. The accompanying sketch clarifies that the trocar sleeve or isolating sleeve exhibits a certain useful length and that the electrode protrudes by a certain measure from the trocar sleeve or from the isolating sleeve. The number of mm by which the electrode protrudes from the trocar sleeve is shown by the so-called adjustment element and is in this case 30 mm. Preferably three different electrode sets are employed, that is a set 50, a set 100, and a set 150, which means that the respective useful length of the trocar sleeve or of the isolating sleeve amounts to 50 mm, 100 mm or 150 mm. The physician has many possibilities of combination when employing different length electrodes, depending on the shape of the tumor and depending on where the tumor is disposed in the body of the patient. Advantageously the sets are distinguished by different surface coloration.

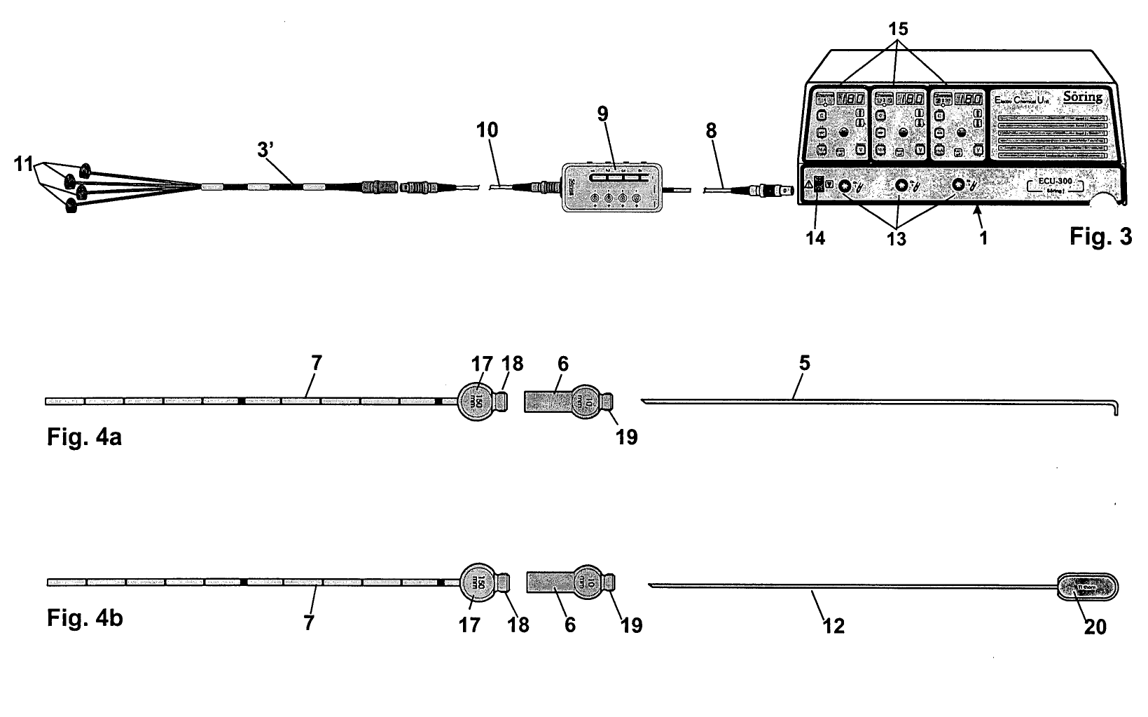

FIG. 3 shows a solution with a switching box (9) disposed between the generator (1) and the instrument cable (3′) for the situation that it becomes necessary during an operating engagement to change the polarity. The output cable (10) leads from the switching box to the instrument cable (3′), wherein the instrument cable (3′) ends with the plugs (11). These plugs are designated with the same symbols (numbers, letters or sign symbols) as is the switching box. Now the surgeon can determine at the switching box (9) with the aid of switches, which switch is to obtain which polarity. The numbers 1 to 4 are printed onto the switching box (9) illustrated in FIG. 3 as a characterization of the coordination to the individual plugs. This means that the surgeon can determine the polarity of the plugs (11) with the aid of these individual switches. The plugs (11) would have been designated also with the numerals 1 to 4 according to this example. And indicator lamp can be coordinated to each switch in the switching box (9) in order to be able to recognize which polarity exhibits in which switch (1 1), that is in for the switch “1” the polarity “+” was elected then the corresponding lamp shines in the switching box (9). This way it can be easily controlled which polarity is set at which plug.

It can be recognized in FIGS. 4a and 4b how the individual parts are joined together. The trocar thorn (12) is shifted into the trocar sleeve (7) and the trocar thorn (12) is screwed onto the thread (18) of the handle part (17) by way of the thread furnished in the handle (20). If it should be required to dispose′ an adjustment element (6,6) between the handle part (17) and the handle (20), then this adjustment element (6,6′) is initially screwed onto the thread (18) of the trocar sleeve (7). Then the handle (20) of the trocar thorn (12) is screwed onto the outer thread (19) of the adjustment element (6,6′). When in operational views then—as already described—the trocar thorn (12) is unscrewed after the placing of the trocar sleeve (7,7′) in the tumor and the electrode (5,5′) is inserted into the trocar shell (7,7′). The electrode (5,5′) is fixed in the adjustment element (6,6′) or, respectively, in the trocar sleeve (7,7′) by the screwed on plug (4,4′, 11).

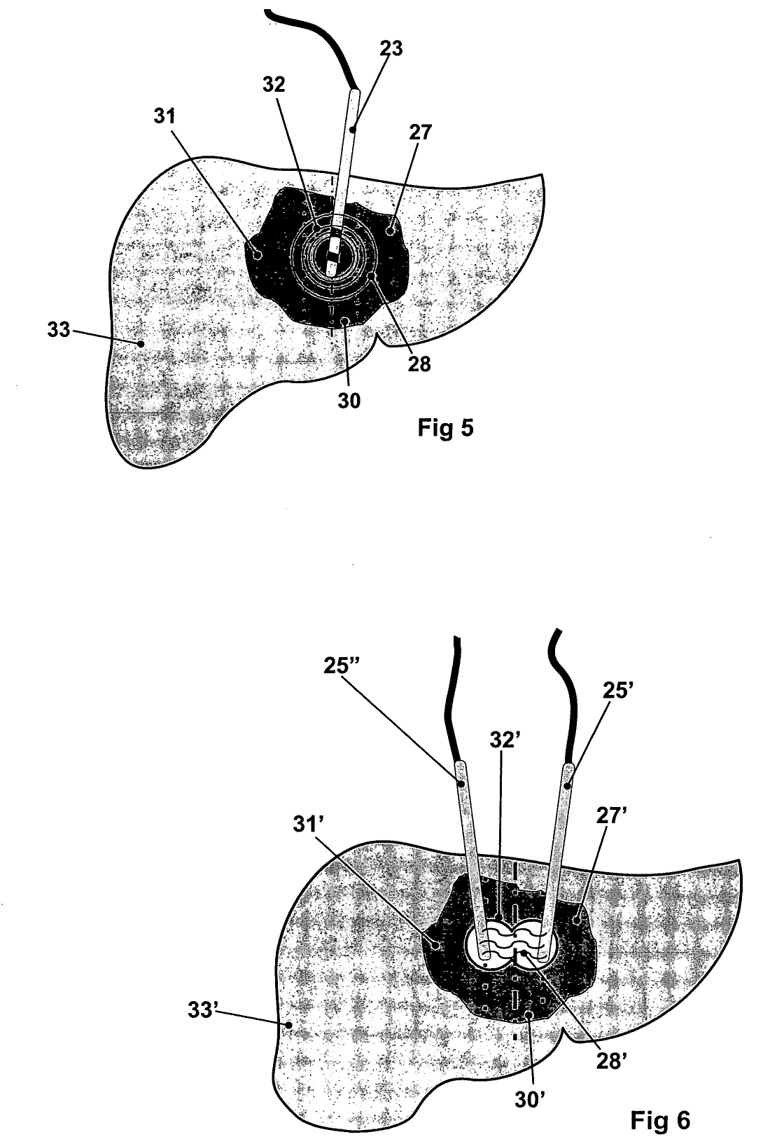

Two different applications are shown in FIGS. 5 and 6 for illustrating the arrangement of the electrodes in connection with the destruction of a tumor in the liver (33). The bipolar electrode (25) is shown in FIG. 5, wherein the bipolar electrode (25) was shifted into the center of a relatively small tumor (30) (size approximately 10 to 20 mm). The tumor (30) is subdivided into three thirds (27,31, 32). The two pole electrode (25) is the such inserted into the center of the tumor (30) such that the active electrode part is disposed in the middle third (32) of the metastases. The current flow is the represented by circles (28).

The single pole electrodes (25′, 25″) are inserted into the tumor (30′) of the liver (33′) at the border of the medium third (32′) with the aid of trocar sleeves in the mono polar electrode arrangement shown in FIG. 6, wherein the mono polar electrode arrangement is employed with tumors in the size of 10 to 50 mm. The active part is adjusted (=about one-third of the diameter of the tumor). The distance between the electrodes (25′, 25″) should amount to at least 15 mm. The current flow (28′) is shown in this example as a wave line.

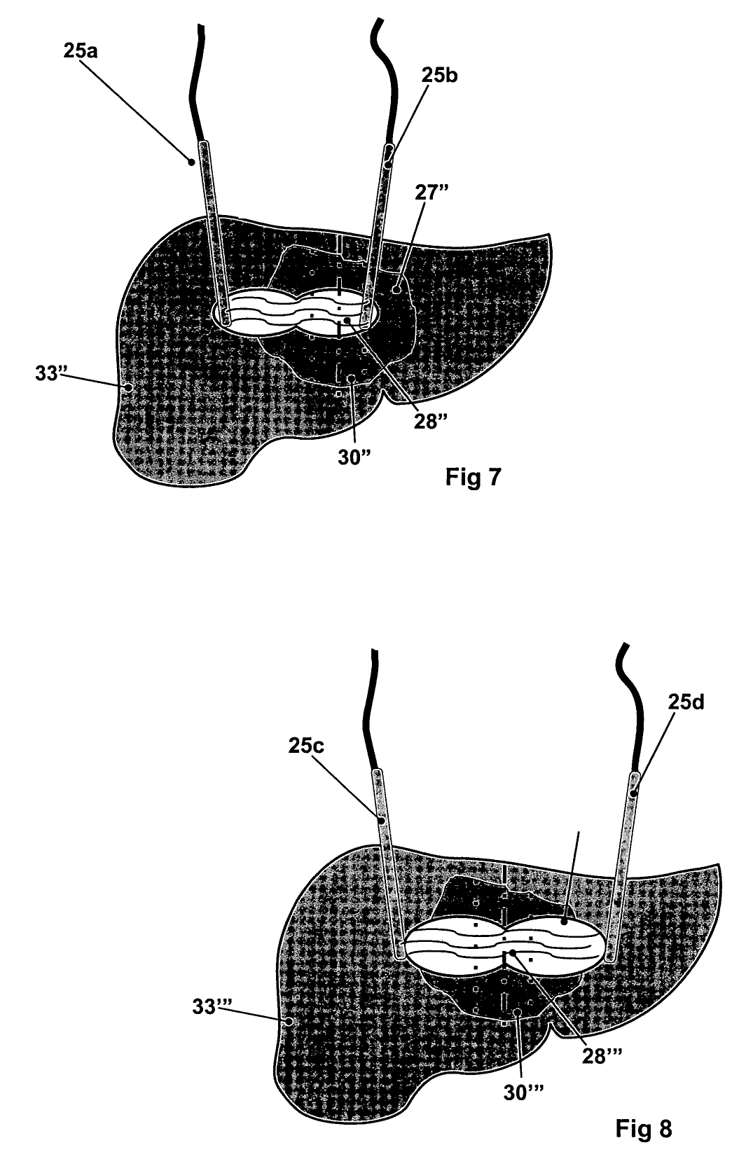

There can be different requirements as to how complete the tumor tissue has to be subjected to necrosis. In case the tumor is to be destroyed completely, it may be advisable to place the first electrode outside of the tumor, but near the tumor into healthy tissue. FIG. 7 shows a liver with a tumor and a polar electrode 25a positioned in the healthy tissue and a second polar electrode 25b positioned in the tumor. FIG. 7 shows an application, where a first electrode 25a is placed into the healthy tissue and where a second electrode 25b is placed inside the tumor at a position relative remote to the position of the first electrode 25a.

FIG. 8 shows a liver with the tumor and two mono polar electrodes 25c, 25d positioned outside of the tumor near two oppositely disposed peripheries of the tumor. FIG. 8 shows an application, where a first electrode 25c is placed into the healthy tissue and where a second electrode 25d is placed into the healthy tissue at a position relative remote to the position of the first electrode 25c on the opposite side of the tumor. Based on the lower resistance of the tumor tissue, the electrical field lines are bundled in the tumor. The positioning of the electrodes according to FIG. 8 can be advantageous where it is essential to destroy the tumor completely and where it can be disregarded that some healthy tissue will also be destroyed.

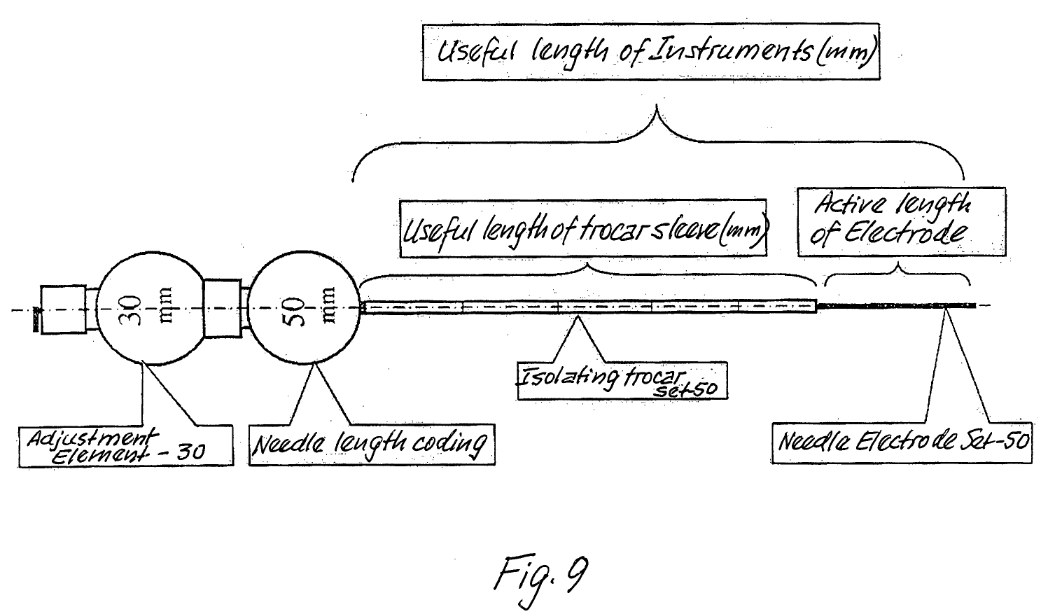

FIG. 9 shows in the center schematicly the trocar extending over the useful length. An active electrode protrudes from the trocar sleeve. Plates are attached to the trocar sleeve end. An adjustment element is mounted on the plate for adjusting the depth of the needle electrode. The diameters of trocar thorn (12), electrode (5) and trocar sleeve (7) or isolating sleeve are dimensions such that there results a slot or an open cylindrical space of from about 0.05 to 0.2 mm and preferably about 0.1 mm between thorn/electrode and the inner diameter of this sleeve. Liquids can be entered or withdrawn or sucked off through this slot or open cylindrical space.

FIG. 9 shows by way of example the construction of an electrode and its support. The needle length of this example is 50 mm. This needle length is stated on the plate disposed at an end of the trocar sleeve 7 disposed remote from the tip of the electrode. A setting element indicating 30 mm is placed on the side of the plate opposite to the trocar sleeve 7 and comprises a rotary ball suitable for adjusting the active electrode length. The useful length of the trocar sleeve is indicated in mm in FIG. 9. The electrode protrudes from the trocar sleeve 7 by a length corresponding to the active electrode length. The active electrode length can be adjusted with the adjustment element 30.

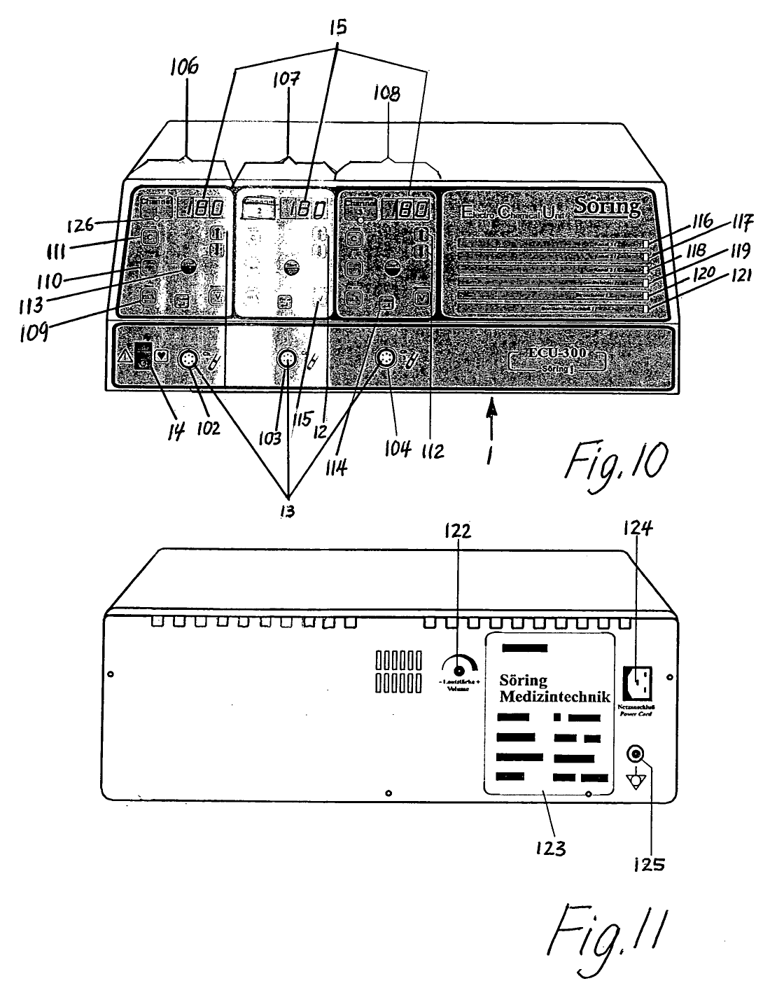

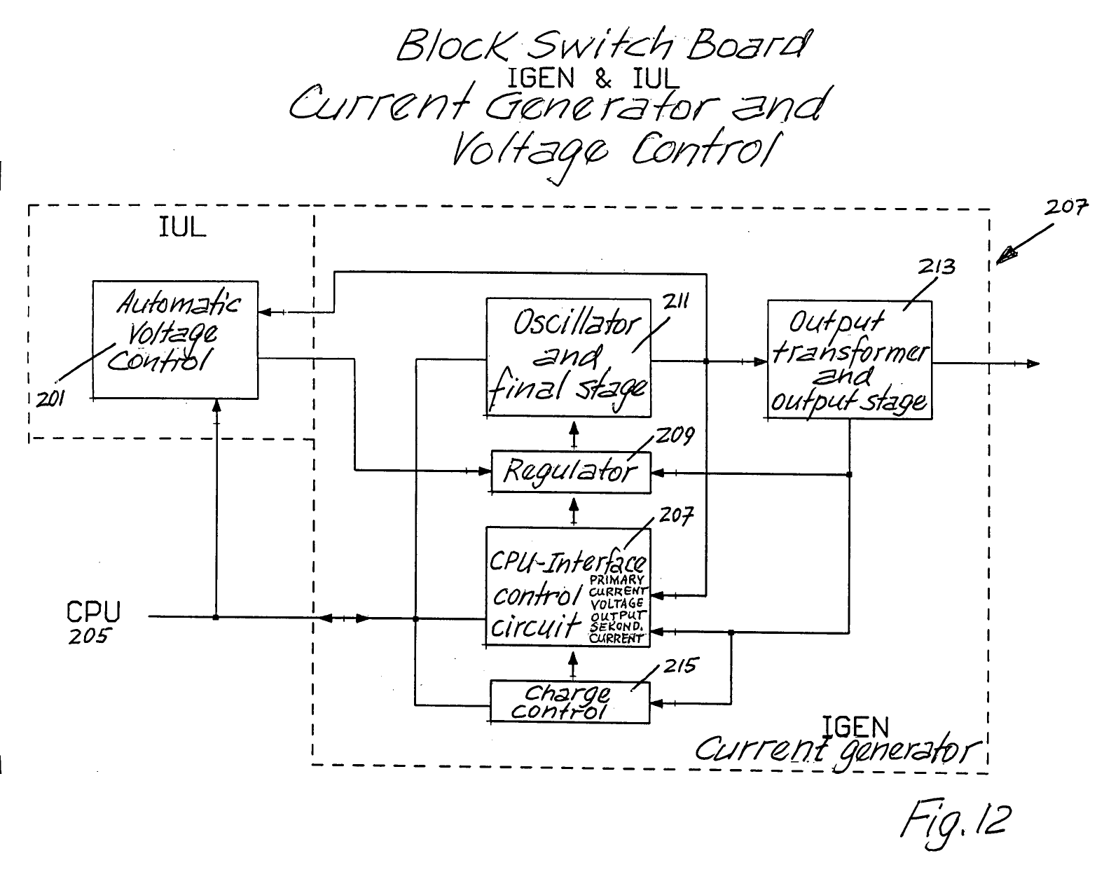

FIG. 10 shows a perspective view of a front panel of a direct current generator. The left-hand side of the current generator is subdivided in three parts each corresponding to a separate channel for providing direct current. The three channels can be operated simultaneously so that it is possible to provide three independent electrode pairs in connection with the tumor removal. The three channels can be operated independently.

The functional unit 15 of FIGS. 3 and 10 offers three channels for the application of special platinum electrodes. Each channel can be the individually adjusted and activated. The treatment is always started or terminated or, respectively, interrupted through the start stop key 113.

FIG. 10 shows the front panel layout of the DC power supply for the electrochemical unit. The power switch 14 is placed on the front panel of the power generator box for turning on electrical power. The first socket channel 102 provides a receptacle for electrical power for the first electrode and is the location, where the electrode cable for the first channel is plugged in. A socket 103 is furnished for the second channel and disposed on the front panel. The electrode cable for the second channel is plugged in the socket 103. A socket 104 is furnished for a third channel. The electrode cable for the third channel is plugged in at the socket 104.

A seven segment display 105 is provided for each of the three channels. The display indicates the value of the selected parameter during setup and the displays the recommended treatment time t during treatment.

The channel 1 function panel is shown at 106. The operating parameters for channel 1 are set out by using the arrow keys 112. The numerical display increments when the ‘arrow up’ key is pressed and decrements when the ‘arrow down’ key is pressed. The value indicated on the display 105 corresponds to the parameter data selected with the keys 109, 110, 111, or 115.

The channel 2 function panel is shown at 107. To operating parameters for channel 2 are set out by using the arrow keys 112. The numerical display increments when the ‘arrow up’ key is pressed and decrements when the ‘arrow down’ key is pressed. The value indicated on the display 105 corresponds to the parameter data selected with the keys 109, 110, 111, or 115.

The channel 3 function panel is shown at 108. To operating parameters for channel 1 are set out by using the arrow keys 112. The numerical display increments when the ‘arrow up’ key is pressed and decrements when the ‘arrow down’ key is pressed. The value indicated on the display 105 corresponds to the parameter data selected with the keys 109, 110, 111, or 115.

The current parameter key ‘mA’ 109 is used to set up the treatment current in mA from about 10 to 120 mA. This key 109 is used to monitor the effective current and the set current during treatment.

The time parameter key ‘t’ 110 is used to set out the entire treatment duration in min. with a maximum of 240 min.. The key 110 is used to monitor the set time during treatment. The recommended time t, the residual time, is continuously displayed during treatment. This residual treatment time cannot be used for time measurement purposes, since the residual time depends on the effective treatment current and the residual charge according to the equation t=Q/I, wherein t is the residual treatment time, Q. is the residual charge, and I is the up-to-date current.

The charge parameter key ‘C’ 111 is used to set up the charge in Coulomb with a maximum of 999 Coulomb. The charge parameter key 111 is used to monitor the residual charge current and to set the charge during treatment.

The up/down arrow key 112 is used to increase or decrease the value of the selected parameter during setup.

The start/stop key 113 is used to start or stop or, respectively, interrupt or continue the treatment.

The enter key 114 is used to select the channel to confirm the set value or to return to the setup mode of the apparatus.

The voltage parameter key 115 is used to set the voltage in volt.

The channel 1 indicator 116 is used to indicate if the channel 1 is defective. The defective channel 1 is also indicated by the current error indicator 120 or probe short circuit indicator 121. A defective channel 1 can also be indicated with the designation ‘Err’ on the display.

The channel 2 indicator 117 is used to indicate if the channel 2 is defective. The defective channel 2 is also indicated by the current error indicator 120 or probe short circuit indicator 121. A defective channel 2 can also be indicated with the designation ‘Err’ on the display.

The channel 3 indicator 118 is used to indicate if the channel 3 is defective. The defective channel 3 is also indicated by the current error indicator 120 or probe short circuit indicator 121. A defective channel 3 can also be indicated with the designation ‘Err’ on the display.

The device error indicator 119 indicates a problem with the device. The device error can be detected during the unit itself test or during operation. In such a case the unit is no longer operable.

The current error indicator 120 indicates a current error if the set current and the effective current, that is the treatment current, differ greatly. The current error indicator 120 indicates a current error with an acoustic and an optical (current error LED) warning if the set current and the effective current (treatment current) differ greatly. The optical warning (flashing indicator) continues as long as the error prevails, while the acoustic warning first sounds permanently and then intermittently. The warning signal remains until the current error has been rectified. When this error is indicated the effective current should be checked as soon as possible. The reason for this deviation can be a high tissue impedance, an incorrect probe placement, a connecting cable interruption, or a defective electrode. When this error occurs, the charge is correctly captured and the time adapted (prolonged or shortened).

The probe short circuit indicator 121 indicates if the probe is shorted for some reason. The indicator flashes if the impedance between the electrodes is very low and the electrodes are short circuited during operation. Then the unit should be checked and the electrodes should be checked. With this error there's the risk that the therapy current does not flow through the tumor but through the probes or within the generator unit. This can indicate an application error, a short circuit event, an electrically conductive path to connect both electrodes, or a defective electrode or a defective electrode cable.

The direct current generator of the Invention device can deliver a settable treatment current of from 10 mA to about 120 mA. The output voltage of the generator can be limited between 25 volts and 5 volts. Preferably a treatment current of from 60 mA to 80 mA is set. Also higher currents for example up to 300 mA are conceivable. The generator allows to set all values of voltage and current and their level of the voltage and current depends on a number of parameters. The proper settings can be found based on the experience of the physician.

The volume of the gases generated by the current at the electrodes 5 depend on the current density. A fixed value is not available since the parameters to be considered can be substantially different.

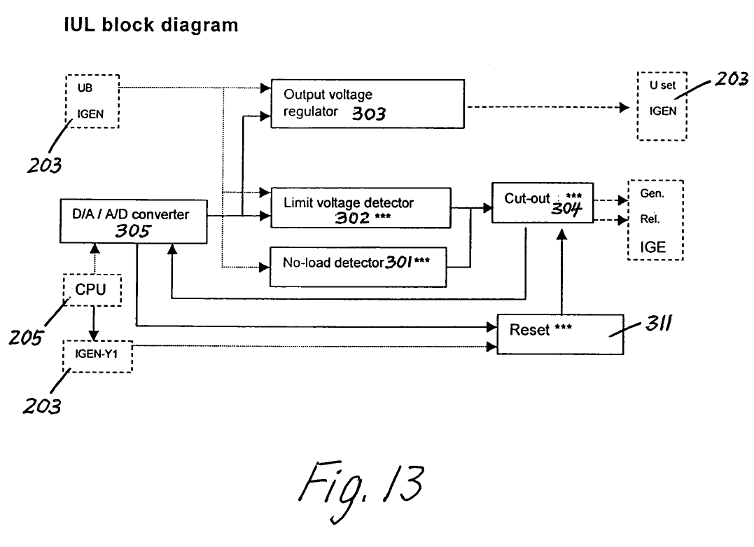

FIG. 11 shows the backside of the direct current generator. The power connection 124 is located on the rear side of the direct current generator. A parameter label 125 is also disposed on the back of the generator 1.The generator case can be grounded through a grounding receptacle 125. The sound strength of various messages and warnings can be adjusted at knob 122 disposed on the rear of the generator case 1.

FIG. 11 shows the back of the apparatus. A volume controller 122 is provided for volume adjustment. The volume controller is employed to adjust the acoustic signal's volume. The volume controller does not affect the volume of the warning signals.

A type label 123 is placed on the generator apparatus giving the ratings, the serial number, the device type, and the fuse rating.

A mains socket 124 is furnished to provide power from the power grid. A power cord is employed to connect the apparatus to a wall socket. The mains socket 124 includes a fuse holder accommodating to main fuses of the device.

An equipotential bonding terminal 125 is also furnished on the rear side of the apparatus the terminal for the equipotential bonding line between the radio frequency device and the operating room's equipotential bonding bar. A yellow green equipotential bonding line is used to make the connection.

An activation light emitting diode 126 is permanently lit when treatment with the respective channel is in progress, when values were set and treatment has not yet been started, or when the user has interrupted the current treatment.

The accompanying block circuit diagram of FIGS. 12, 13, and 14 shows the construction of the generator. Each of the channels 1, 2, 3, is furnished with an alike, but a separate circuit arrangement according to FIGS. 12, 13, and 14.

FIG. 12 shows a block circuit diagram with the current direct current generator IGEN and the automatic voltage control IUL. The first input of the automatic voltage control 201 comes from the central processing unit 205. The automatic voltage control 201 is further connected to a primary current output of a CPU interface control circuit 207 of the direct current generator 203. An output of the automatic voltage control is connected to an input of an automatic controller 209. Furthermore the central processing unit 205 is connected to the central processing unit interface control 207. An output of the CPU interface control circuit is connected to the automatic controller 209. The output of the automatic controller 209 is directed to an oscillator and final stage 211. The oscillator and final stage 211 has an output connected to an input of an output transformer and output stage 213. The output transformer and output stage 213 is connected to the electrodes. The feedback output of the output transformer and output stage 213 is connected to an input of a charge control 215, to a secondary current input of the central processing unit interface control 207 and to the automatic controller 209. The charge control 215 has an output connected to the central processing unit interface control 207 and is further connected to the oscillator and final stage 211.

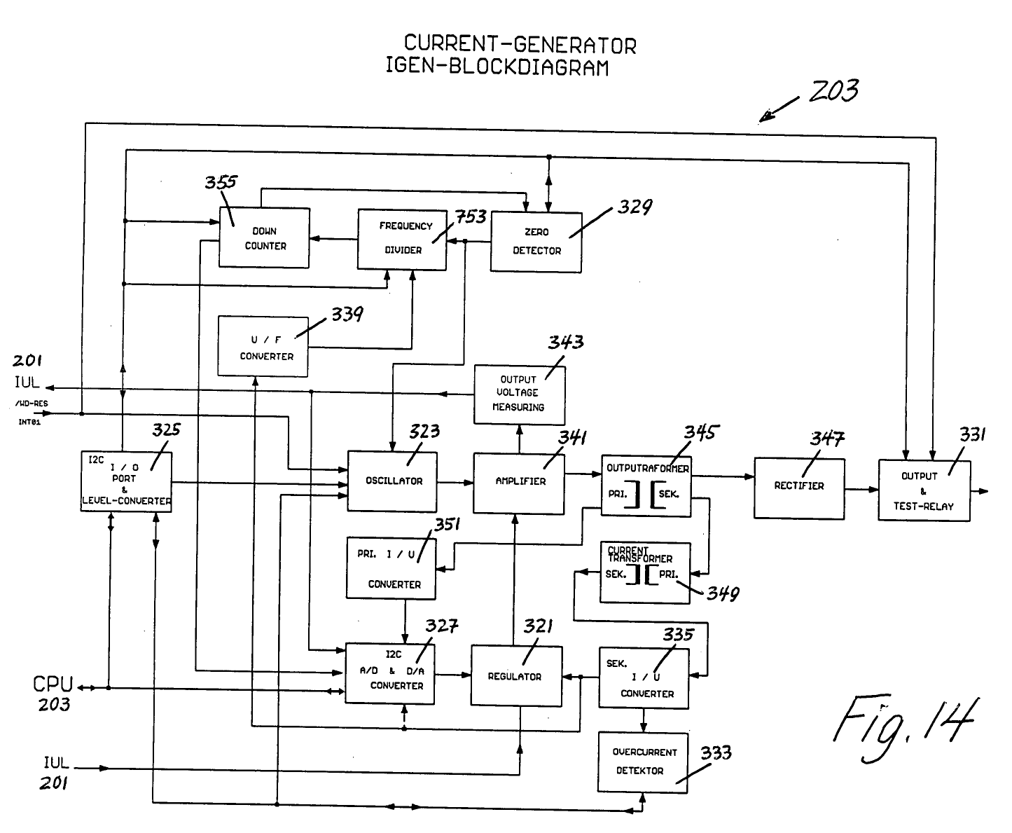

FIG. 13 shows a block diagram of the automatic voltage control. The CPU has an output connected to an input of a digital to analog and analog to digital converter 305. The digital analog/analog digital converter 305 has an output connected to an input of a reset 311. Furthermore, the central processing unit 205 is connected to the direct current generator 203 and from the direct current generator to the reset circuit 311. The output of the reset circuit 311 is connected to an input of cut-out circuit 304. An output of the cut out circuit 304 is connected to an input of the digital to analog/analog to digital converter 305.

A second output of the digital to analog and analog to digital converter 305 is connected to an input of a limit voltage detector 302 and to an input of an output voltage regulator 303.

A specific voltage UB of the direct current generator 203 is fed to an input of the output voltage regulator 303, to an input of the limit voltage detector 302, and to an input of the no-load detector 301 as shown in FIG. 13. The output of the limit voltage detector 302 and the output of the no-load detector 301 are fed to a second input of the cut out circuit 304. The output of the cut out circuit 304 is fed to a general relay or to a regulator 321. The output of the output voltage regulator 303 is fed to an input of the direct current generator 203 for the set point voltage Uset.

An output of the automatic voltage control circuit 201 is fed to an input of the regulator 321 of the direct current generator 203 according to FIG. 14. A second output of the automatic voltage controller 201 is fed to the oscillator 323 of the direct current generator 203 and to the output and test relay circuit 331.

The central processing unit 205 is connected to an I/O port and level converter 325 and to an analog to digital and digital to analog converter 327. The I/O port and level converter 325 is connected to an input of a down counter 355, to a frequency divider 353, to a zero detector 329, and to an input of an output and test relay circuit 331. The I/O port and level converter 325 is connected to the over current detector 333. A

The analog to digital and digital to analog converter 327 has an output connected to an input of the automatic control 321. The I/O port and level converter 325 is further connected to the oscillator 323, to zero detector 329, to an output and test relay 331, and to an over current detector 333. A secondary current to voltage converter 335 has an output connected to the over current detector 333 and a second output connected to the regulator 321 and to a voltage to frequency converter 339 and to an input of the analog to digital and digital to analog converter 327. The oscillator 323 and the regulator 321 each have an output connected to an input of an amplifier 341. The amplifier 341 has an output connected to an input of an output voltage measuring circuit 343. The output voltage measuring circuit 343 has an output connected to an input of the automatic voltage controller 201 and to an input of the analog to digital and digital to analog converter 327.

A second output of the amplifier 341 is connected to an output transformer 345. The output transformer 345 has an output connected to an input of the rectifier 347. The rectifier 347 has an output connected to an input of the output and test relay circuit 331. A second output of the output transformer 345 is fed to an input of a current transformer 349. An output of the current transformer 349 fed to an input of the secondary current to voltage converter 335. The third output of the output transformer 345 is connected to an input of a primary current to voltage converter 351. An output of the primary current to voltage converter 351 is fed to an input of the analog to digital and digital to analog converter 327.

An output of the voltage to frequency converter 339 is fed to an input of the frequency divider 353. An output of the Zero detector 329 is fed to an input of the frequency divider 353. An output of the frequency divider 353 is fed to an input of a down counter 355. A first output of the down counter 355 is fed to an input of the zero detector 329. A second output of the down counter 355 is fed to an input of the analog to digital and digital to analog converter 327.

Mode of Operation

The treatment is always started or terminated or, respectively, interrupted through the start stop key 113 associated with a respective one of the three channels. The physician individually adjusts and activates each channel.

The trocar sleeve and the adjustment element are connected initially to each other by the physician.

The physician can apply the electrodes percutaneously under local anaesthesis. The fitting trocar is led up to the tumor for this purpose.

The physician selects the length of the electrodes depending on the field of application. The physician determines the location of the tumor in the body, and the corresponding trocar sleeve (7) and coordinated trocar thorn (12) and needle electrode (5) are selected of an appropriate length. The length of the free electrode part is determined by the physician based on the size or, respectively, the thickness of the tumor.

Then following the trocar thorn (12) is the slipped through the trocar sleeve (7) in order to stitch an opening into the tissue. Thereafter the trocar thorn (12) is pulled out and the needle electrode (5) is inserted into the stiched opening.

The electrode is then positioned in the trocar. The positioning of the electrodes should be continuously monitored through for example ultrasound instruments.

The physician uses the adjustment element for adjusting the part of the needle electrode protruding from the trocar sleeve. The adjustment element practically represents a spacer piece. If, for example, the needle electrode is to protrude by 10 mm from the front and of the trocar sleeve, then the 10 mm adjustment element is inserted; if the electrode is to protrude by 30 mm, then the 30 mm element is employed. The physician can combine various elements such as employing different length electrodes, depending on the shape of the tumor and depending on where the tumor is disposed in the body of the patient. The physician can distinguish the sets by different surface coloration.

A special securing of the trocar sleeve (5) is not required. The physician observes the markings at the trocar sleeve (7) and holds the trocar sleeve (7) with one hand by using the other hand for changing the electrode (5) or the trocar thorn (12).

FIGS. 4a and 4b show that the trocar sleeve and the adjustment element are connected initially to each other. Then following the trocar thorn (12) is slipped through the trocar sleeve (7) in order to stitch an opening into the tissue. Thereafter the trocar thorn (12) is pulled out and the needle electrode (5) is inserted.

As shown in FIG. 3, a switching box (9) can be placed between the generator (1) and the instrument cable (3′) for whenever it might become necessary during an operating engagement to change the polarity. The output cable (10) leads from the switching box to the instrument cable (3′), wherein the instrument cable (3′) ends with the plugs (11). These plugs are designated with the same symbols (numbers, letters or sign symbols) as is the switching box. Now the surgeon can determine at the switching box (9) with the aid of switches, which switch is to obtain which polarity. The numbers 1 to 4 are printed onto the switching box (9) illustrated in FIG. 3 as a characterization of the coordination to the individual plugs. This means that the surgeon can determine the polarity of the plugs (11) with the aid of these individual switches. The plugs (11) would have been designated also with the numerals 1 to 4 according to this example. An indicator lamp can be coordinated to each switch in the switching box (9) in order to be able to recognize which polarity exhibits in which switch (11), that is in for the switch “1” the polarity “+” was elected then the corresponding lamp shines in the switching box (9). This way it can be easily controlled which polarity is set at which plug.

The parameters employed have to be predetermined and estimated by the physician prior to treatment. The setting of the parameters (charge, current, time and output voltage) are depending on the tumor size, the tumor kind, the tumor consistency and the like. The generator is switched off after the preset charge volume (Q=D tumor×100) (D in cm) has been delivered. The number of the electrodes employed determines the voltage and the current.

The electrodes are preferably employed in the direction of the largest extension of the tumor if possible. In case other members and the bones cover the tumor then another way has to be employed for insertion of the electrodes.

In case the tumor is disposed deeply in the body of the patient and also is relatively large, then the Invention method can also be performed step-by-step. This means that the physician shifts in the trocar sleeve or isolating sleeve first to the extreme necessary depth and then pulls sleeve and electrode backward step-by-step.

The joining of the individual parts is shown in FIGS. 4a and 4b. The trocar thorn (12) is shifted into the trocar sleeve (7) and the trocar thorn (12) is screwed onto the thread (18) of the handle part (17) by way of the thread furnished in the handle (20). If it should be required to dispose' an adjustment element (6,6) between the handle part (17) and the handle (20), then this adjustment element (6,6′) is initially screwed onto the thread (18) of the trocar sleeve (7). Then the handle (20) of the trocar thorn (12) is screwed onto the outer thread (19) of the adjustment element (6,6′). When in operational views then—as already described—the trocar thorn (12) is unscrewed after the placing of the trocar sleeve (7,7′) in the tumor and the electrode (5,5′) is inserted into the trocar shell (7,7′). The electrode (5,5′) is fixed in the adjustment element (6,6′) or, respectively, in the trocar sleeve (7,7′) by the screwed on plug (4,4′, 11).

The parameters required for the treatment can be separately adjusted for the individual connection bushings. Depending on the case of application—as can be recognized from FIG. 2—two electrodes (5,5′) can be connected to the connection bushings of the generator (1) by way of the connection cable (2) and the plug attached thereto. The instrument cable (3) is connected to this connection cable (2), wherein the plugs (4,4′) are attached at the other end of the instrument cable (3). These plugs (4,4′) are electricly conducting connected to the electrodes after the insertion and the precise fixation of the trocar sleeves (7,7′) and after the insertion of the electrodes (5,5′) into the respective trocar sleeve (7,7′). Depending on which electrode length is available, an adjustment element (6,6′)′ can be disposed between the trocar sleeve (7,7′) and the plug (4,4′).

The physician aligns the electrodes under ultrasonic control or under MRT-or CT-monitoring.

The physician can pre-drill a hole with the biopsy set and then can employ the trocar sleeve 7 or isolating sleeve.

The physician can feed liquids into the tumor through the trocar sleeve 7.

The physician inserts the needle electrode into the trocar sleeve through the adjustment element (6) and the handle part (17).

Haemostasis or blood stanching is only required, in case larger vessels are injured. Haemostasis can be performed for example by coagulation with the electrode pulled out of the trocar sleeve or isolating sleeve, then applying voltage to the electrode and thereby performing an electro-coagulation. The physician determines if blood stanching is required and if appropriate performs electro-coagulation of the blood in question.

The treatment time can be freely selected. For electrolysis the current I, the charge Q, and the time t are set, and therein relationship Q=I×t holds. The maximum treatment time is 240 min. the maximum preset charge is 999 Coulomb.

The completeness of electrolysis is determined by a control with ultrasound, computer tomogram CT, or MRT immediately after the treatment and for control purposes another time a day later.

It will be understood that each of the elements described above, or two or more together, may also find a useful application in other types of electrolytic system configurations and tumor removal procedures differing from the types described above.

While the invention has been illustrated and described as embodied in the context of an instrument for the destruction of tumor tissues, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

### LIST OF REFERENCE NUMERALS

1—generator

2—connection cable

3,3′—instrument cable

4,4′—plug

5,5′—electrode

6,6′—adjustment element

7,7′—trocar sleeve

8—connection line

9—switching box

10—output cable

11—plug

12—trocar thorn

13—connection bushing

14—power switch

15—function units

16—

17—handle part

18—thread

19—outer thread

20—handle

25, 25′, 25″, 25a, 25b, 25c, 25d—electrodes

26—

27—3rd tumor third

28, 28′—current flow

29—

30,30′—tumor

31,31′—first tumor third

32, 32′—second tumor third

33,33′—liver

102—first socket channel

103—socket

104—socket

105—seven segment display

106—channel 1 function panel

107—channel 2 function panel

108—channel 3 function panel

109—current parameter key ‘mA’

110—time parameter key ‘t’

111—charge parameter key ‘C’

112—up/down arrow key

113—start stop key

114—enter key

115—voltage parameter key

116—channel 1 indicator

117—channel 2 indicator

118—channel 3 indicator

119—device error indicator

120—current error indicator

121—probe short circuit indicator

122—sound volume controller

123—model and parameter label

124—power connection

125—grounding receptacle

201—automatic voltage control

203—direct current generator

205—central processing unit

207—CPU interface control circuit

209—automatic controller

210

211—oscillator and final stage

212

213—output transformer and output stage

214

215—charge control

301—no-load detector

302—limit voltage detector

303—output voltage regulator

304—cut-out circuit

305—digital to analog and analog to digital converter

321—regulator

331—oscillator

325—IO port and level converter

327—an analog to digital and digital to analog converter

328—zero detector

331—output and test relay circuit

333—over current detector

335—secondary current to voltage converter

339—voltage to frequency converter

341—amplifier

343—output voltage measuring circuit

345—output transformer

347—rectifier

349—current transformer

351—primary current to voltage converter

353—frequency divider

355—down counter

1. An instrument for electrochemical treatment of the human body or of an animal body, in particular for the destruction of tumor tissues with one or several electrodes for insertion into the body to be treated, characterized in that this instrument comprises
a generator (1) generating a DC voltage,
connection lines (2,3,3′),
at least one trocar sleeve′ (7,7),
at least one trocar thorn (12), and
at least one electrode (5,5′).

2. The instrument for the electrochemical treatment of the human body or of an animal body according to claim 1, characterized in that an adjustment element (6,6′) is furnished between the instrument cable (3,3′) and the trocar sleeve (7,7′).

3. The instrument for the electrochemical treatment of the human body or of an animal body according to claim 1 characterized in that plugs (4,4′, 11, 11′) of different polarity are disposed at the end of the instrument cable.

4. The instrument for the electrochemical treatment of the human body or of an animal body according to claim 1 characterized in that a trocar thorn (12) can be inserted into the trocar sleeve (7,7′).

5. The instrument for the electrochemical treatment of the human body or of an animal body according to claim 1 characterized in that the electrode (5,5′) is made out of platinum.

6. The instrument for the electrochemical treatment of the human body or of an animal body according to claim 1 characterized in that trocar thorn (12) is made out of stainless-steel.

7. The instrument for the electrochemical treatment of the human body or of an animal body according to claim 6 characterized in that the trocar thorn (12) is covered with the non-adhering material, for example poly tetra fluoro ethylene.

8. An instrument for the electrochemical treatment of the human body or of an animal body, in particular for the destruction of tumor tissues with one or several electrodes for insertion into the body to be treated characterized in that this instrument comprises
a generator (1) generating a DC voltage,
connection lines (2,3,3′),
a switching box (9) for the selection of the polarity,
at least one trocar sleeve (7,7′),
at least one trocar thorn (12), and
at least one electrode (5,5′).

9. The instrument for the electrochemical treatment of the human body or of an animal body according to claim 8 characterized in that the instrument cables (3′) connected to the switching box (9) are not furnished with a fixed polarity.

10. The instrument for the electrochemical treatment of the human body or of an animal body according to claim 8 characterized in that the plug (11) of the instrument cable is designated in color.

11. The instrument for the electrochemical treatment of the human body or of an animal body according to claim 8, characterized in that the plugs ( 1) of the instrument cable (3,3′) are designated with numerals or letters.

12. The instrument for the electrochemical treatment of the human body or of an animal body according to claim 10, characterized in that the designations of the plugs (11) of the instrument cable (3′) are congruent to the designations at the switching box.

13. The instrument for the electrochemical treatment of the human body or of an animal body according to claim 1, characterized in that the generator (1) for generating a DC voltage is furnished with several separately automatically controllable outputs (13).

14. The instrument for the electrochemical treatment of the human body or of an animal body according to claim 1, characterized in that various parameters such as charging, time, current, and voltage as well as the starting of the treatment can be adjusted and set at the generator (1).

15. A method for the electrochemical treatment of the human body or of an animal body, in particular for the destruction of tumor tissues with one or several electrodes for insertion into the body to the treated characterized in that at least one or several bipolar or mono polar electrodes (5,5′) can be inserted into a tumor and then can be subjected to a DC voltage.

16. The method for the electrochemical treatment of the human or of an animal body according to claim 15 characterized in that tumor is necrotized by the electrolysis induced by the DC voltage.

17. The method for the electrochemical treatment of the human body or of an animal body according to claim 15 characterized in that this method is employed in minimally invasive operations.

18. The method for the electrochemical treatment of the human body or of an animal body according to claim 15 characterized in that this method is employed with open operations.

19. Instrument or method for the electrochemical treatment of the human body or of an animal body according to claim 1 characterized in that the trocar sleeve (7,7′), the trocar thorn (12), and the electrode (5,5′) can be subjected to treatment by an autoclave.

20. An instrument for electrochemical treatment of the human body or of an animal body comprising
a generator (1) generating a DC voltage;
connection lines (2,3,3′) connected to the generator;
a trocar sleeve′ (7,7) connected to the connection lines;
a trocar thorn (12) insertable into the trocar sleeve; and
an electrode (5,5′) passing through the trocar sleeve and protruding from the end of the trocar sleeve and directing electrical currents toward a tumor for destructing the tumor.

21. The instrument for the electrochemical treatment of the human body or of an animal body according to claim 20, further comprising
an adjustment element (6,6′) furnished between the connection lines (3,3′) and the trocar sleeve (7,7′);
plugs (4,4′, 11, 11′) of different polarity disposed at an end of the connection lines (3,3′);
wherein the electrode (5,5′) is made out of platinum; and
wherein the trocar thorn (12) is made out of stainless-steel.
wherein the trocar thorn (12) is covered with the non-adhering material, for example poly tetra fluoro ethylene.

22. An instrument for the electrochemical treatment of the human body or of an animal body, in particular for the destruction of tumor tissues comprising
a generator (1) generating a DC voltage,
connection lines (2,3,3′) connectable to the generator;
a switching box (9) inserted into the connection lines for a selection of polarity;
at least one trocar sleeve (7,7′) connected with a first end to the connection lines;,
at least one trocar thorn (12) insertable into the trocar sleeve, and
at least one electrode (5,5′) insertable into the trocar sleeve such that an end of the electrode (5,5′) protrudes from a second end of the trocar sleeve.

23. The instrument for the electrochemical treatment of the human body or of an animal body according to claim 22 further comprising
a plug (11) disposed at an end of the connection lines and having a designation in color and wherein the plugs (11) of the connection lines (3,3′) are designated with numerals or letters wherein the designations of the plugs (11) of the connection lines (3′) are congruent to designations at the switching box;;
wherein the connection lines (3′) connected to the switching box (9) are capable of changing their polarity by switching the switching box;
wherein the generator (1) for generating a DC voltage is furnished with several separately automatically controllable outputs (13).
wherein various parameters such as charging, time, current, and voltage as well as the starting of the treatment can be adjusted and set at the generator (1).

24. A method for the electrochemical treatment of the human body or of an animal body, comprising the steps of:
inserting an electrode into the body in an area of a tumor;
charging the electrode with a direct current;
subjecting the area of the tumor to electrolysis.;
effecting a destruction of tumor cells by changing electro-chemical potentials in a direction for not sustaining tumor cells, and
destroying tumor tissues with the electrolysis products.

25. The method for the electrochemical treatment of the human or of an animal body according to claim 24 further comprising
necrotizing the tumor by the electrolysis induced by the DC voltage;
performing the method as a minimally invasive operation or as an open operation.

26. The instrument for the electrochemical treatment of the human body or of an animal body according to claim 22 wherein the generator further comprises
a front panel;
a start stop key (113) disposed on the front panel for starting or terminating or, respectively, interrupting the treatment;.
a power switch (14) placed on the front panel of the generator for turning on electrical power;
a first socket channel (102) having a receptacle for electrical power for the first electrode and thereby a location, where the connection line for the first channel is plugged in;
a seven segment display (105) disposed on the front panel and indicating a value of a selected parameter during setup and displaying a recomended treatment time t during treatment;
up/down arrow keys (112) disposed on the front panel for setting the operating parameters;
a current parameter key ‘mA’ (109) disposed on the front panel for setting up a treatment current in mA from about 10 to 120 mA;
a time parameter key ‘t’ (110) disposed on the front panel for setting out the entire treatment duration in min. with a maximum of 240 min;
a charge parameter key ‘C’ (111) disposed on the front panel for setting up the charge in Coulomb with a maximum of 999 Coulomb;
an enter key (114) disposed on the front panel for selection of a channel, for confirming a set value or for returning to the setup mode of the apparatus.
a voltage parameter key (115) disposed on the front panel and employed to set a voltage in volt;
an indicator (116) disposed on the front panel for indicating if the channel is defective;
a device error indicator (119) disposed on the front panel and indicating a problem with the generator;
a current error indicator (120) disposed on the front panel and indicating a current error if the set current and the effective current, that is the treatment current, differ greatly; and
a probe short circuit indicator (121) disposed on the front panel and indicating if the probe is shorted for some reason.

27. The instrument for the electrochemical treatment of the human body or of an animal body according to claim 22 wherein the generator further comprises
a rear panel;
a power connection (124) is located on the rear panel of the generator;
a volume controller (122) disposed on the rear panel is furnished for manual volume adjustment; and
an equipotential bonding terminal (125) disposed on the rear panel, wherein the terminal is for an equipotential bonding line between the generator and the operating room's equipotential bonding bar.

28. The instrument for the electrochemical treatment of the human body or of an animal body according to claim 22 wherein the generator further comprises
an automatic voltage control (201);
a current direct current generator (203) including
a CPU interface control circuit (207), wherein the automatic voltage control (201) is connected to a primary current output of a CPU interface control circuit (207) of the direct current generator (203);
an automatic controller (209), wherein an output of the automatic voltage control is connected to an input of an automatic controller (209), wherein a central processing unit (205) is connected to the central processing unit interface control (207), and wherein an output of the CPU interface control circuit is connected to the automatic controller (209);
an oscillator and final stage (211), wherein an output of the automatic controller (209) is directed to the oscillator and final stage (211);
an output transformer and output stage (213), wherein the oscillator and final stage (211) has an output connected to an input of an output transformer and output stage (213) and wherein the output transformer and output stage (213) is connected to the electrode;
a charge control (215), wherein a feedback output of the output transformer and output stage (213) is connected to an input of the charge control (215), to a secondary current input of the central processing unit interface control (207) and to the automatic controller (209), and wherein the charge control (215) has an output connected to the central processing unit interface control (207) and is further connected to the oscillator and final stage (211).

29. The instrument for the electrochemical treatment of the human body or of an animal body according to claim 22 wherein the generator further comprises
a central processing unit (205);
a digital to analog and analog to digital converter (305), wherein the central processing unit (205) has an output connected to an input of a digital to analog and analog to digital converter (305);
a reset circuit (311), wherein the digital analog/analog digital converter (305) has an output connected to an input of the reset circuit (311);
a direct current generator (203), wherein the central processing unit (205) is connected to the direct current generator (203) and from the direct current generator to the reset circuit (311);
a cut-out circuit (304), wherein the output of the reset circuit (311) is connected to an input of the cut-out circuit (304), and wherein an output of the cut out circuit (304) is connected to an input of the digital to analog/analog to digital converter (305);
a limit voltage detector (302);
an output voltage regulator (303), wherein a second output of the digital to analog and analog to digital converter (305) is connected to an input of the limit voltage detector (302) and to an input of the output voltage regulator (303);
a no-load detector (301), wherein a specific voltage UB of the direct current generator (203) is fed to an input of the output voltage regulator (303), to an input of the limit voltage detector (302), and to an input of the no-load detector (301), and wherein the output of the limit voltage detector (302) and the output of the no-load detector (301) are fed to a second input of the cut out circuit (304), and wherein an output of the cut out circuit (304) is fed to a general relay or to a regulator (321), and wherein an output of the output voltage regulator (303) is fed to an input of the direct current generator (203) for the set point voltage Uset.

30. The instrument for the electrochemical treatment of the human body or of an animal body according to claim 22 wherein the generator further comprises
a regulator (321);
an automatic voltage control circuit (201), wherein an output of the automatic voltage control circuit (201) is fed to an input of the regulator (321) of a direct current generator (203);
an oscillator (323);
an output and test relay circuit (331), wherein a second output of the automatic voltage controller (201) is fed to the oscillator (323) of the direct current generator 203 and to the output and test relay circuit (331);
a central processing unit (205);
an I/O port and level converter (325);
an analog to digital and digital to analog converter (327), wherein the central processing unit (205) is connected to the I/O port and level converter (325) and to the analog to digital and digital to analog converter (327);
a down counter (355);
a frequency divider (353);
a zero detector (329), wherein the I/O port and level converter (325) is connected to an input of the down counter (355), to the frequency divider (353), to the zero detector (329), and to an input of the output and test relay circuit (331);
an over current detector (333), wherein the I/O port and level converter 325 is connected to the over current detector (333),
wherein the analog to digital and digital to analog converter (327) has an output connected to an input of the automatic control (321),
wherein the I/O port and level converter (325) is further connected to the oscillator (323), to zero detector (329), to the output and test relay circuit (331), and to the over current detector (333);
a voltage to frequency converter (339);
a secondary current to voltage converter (335) having an output connected to the over current detector (333) and a second output connected to the regulator (321) and to the voltage to frequency converter (339) and to an input of the analog to digital and digital to analog converter (327);
an amplifier (341), wherein the oscillator (323) and the regulator (321) each have an output connected to an input of the amplifier (341);
an output voltage measuring circuit (343), wherein the amplifier (341) has an output connected to an input of an output voltage measuring circuit (343), and wherein the output voltage measuring circuit (343) has an output connected to an input of the automatic voltage controller (201) and to an input of the analog to digital and digital to analog converter (327);
an output transformer (345), wherein a second output of the amplifier (341) is connected to the output transformer (345);
a rectifier (347), wherein the output transformer (345) has an output connected to an input of the rectifier (347), and wherein the rectifier (347) has an output connected to an input of the output and test relay circuit (331);
a current transformer (349), wherein a second output of the output transformer (345) is fed to an input of the current transformer (349), and
wherein an output of the current transformer (349) is fed to an input of the secondary current to voltage converter (335);
a primary current to voltage converter (351), wherein a third output of the output transformer (345) is connected to an input of the primary current to voltage converter (351), and wherein an output of the primary current to voltage converter (351) is fed to an input of the analog to digital and digital to analog converter (327), wherein an output of the voltage to frequency converter (339) is fed to an input of the frequency divider (353), wherein an output of the Zero detector (329) is fed to an input of the frequency divider (353), wherein an output of the frequency divider (353) is fed to an input of the down counter (355), wherein a first output of the down counter (355) is fed to an input of the zero detector (329), and wherein a second output of the down counter (355) is fed to an input of the analog to digital and digital to analog converter (327).
